(19) United States
(12) Patent Application Publication
(10) Pub. No.: US 2026/0248771 A1
(43) Pub. Date: Aug. 27, 2026

(54) VALBENAZINE, A VMAT2 INHIBITOR, AS A FREE BASE A TOSYLATE OR DITOSYLATE SALT, FOR USE IN THE TREATMENT OF CHOREA ASSOCIATED WITH HUNTINGTON'S DISEASE

(71) Applicant: Neurocrine Biosciences, Inc., San Diego, CA (US)

(72) Inventors: Dietrich Haubenberger, San Diego, CA (US); Grace S. Liang, San Diego, CA (US)

(21) Appl. No.: 18/844,531

(22) PCT Filed: Mar. 3, 2023
(86) PCT No.: PCT/US2023/063663

§ 371 Date: Sep. 6, 2024

Related U.S. Application Data
(60) Provisional application No. 63/268,937, filed on Mar. 7, 2022.
(60) Provisional application No. 63/327,280, filed on Apr. 4, 2022.
(60) Provisional application No. 63/419,224, filed on Oct. 25, 2022.

(51) Int. Cl. A61K 31/4375; A61P 25/14

(52) U.S. Cl. CPC A61K 31/4375; A61P 25/14

(57) ABSTRACT

Provided herein are methods for treatment of chorea associated with Huntington disease using a vesicular monoamine transporter isoform 2 (VMAT2) inhibitor. In certain embodiments, the VMAT2 inhibitor is valbenazine or a pharmaceutically acceptable salt thereof.

### BACKGROUND

Huntington disease (HD) is a genetic progressive neurodegenerative disease characterized clinically by chorea, cognitive dysfunction, and psychiatric symptoms. Pathologically, HD is associated with brain atrophy characterized by loss of striatal medium spiny neurons and cortical pyramidal neurons. Neurochemically, alterations in dopamine function and neurotransmission is observed in HD patients, with increased dopamine neurotransmission observed with early stage HD symptoms manifesting as hyperkinetic abnormal involuntary movements. Later stage motor symptoms are primarily hypokinetic in nature.

Patients with HD typically have onset of symptoms at approximately 30 to 50 years of age. Five to 10% of cases are classified as juvenile onset, with patients becoming symptomatic before the age of 20. The average lifespan after symptom onset is 15 to 20 years.

One of the defining motor symptoms of HD is chorea, characterized as abnormal, abrupt, irregular, nonstereotyped movements. As HD progresses, chorea can increase in frequency and amplitude. Chorea is also associated with decreased quality of life. While there is no established treatment to delay onset or progression of HD, treatment of HD-associated chorea could benefit some patients through improved quality of life.

### BRIEF SUMMARY

Provided is a method for the treatment of chorea associated with Huntington disease, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof.

These and other aspects of the disclosure will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF THE DRAWINGS

FIG. 1 shows a schematic of the study design. The study includes a Screening Period, a Dose-Adjustment Period, and a Maintenance Period.

FIG. 2 shows a summary of participants in the study described in Example 1. At baseline, subjects were balanced between treatment groups in terms of demographics and disease characteristics. The chorea severity at baseline was similar between groups.

FIG. 3 shows a summary of primary endpoints from the study described in Example 1. The primary endpoint of change of total maximal chorea (TMC) score from the screening-baseline period to the maintenance period, was highly statistical significant, indicating greater improvement of chorea symptom severity in valbenazine treated subjects, compared to placebo. Least-squares mean change is shown for placebo and valbenazine groups, where N=61 (placebo), N=64 (valbenazine). P values are derived from using a MMRM analysis on the full analysis set. Bars indicate one standard error.

FIG. 4 shows mean change in chorea symptoms from screening period baseline by study visit from the study described in Example 1. Least-squared mean change from screening period baseline was graphed as a function of time via weekly study visit time points, from baseline and every two weeks until week 12 from initial baseline visit. Error bars shown are standard deviation, where N=61 (placebo), N=64 (valbenazine) for each time point after baseline period. P values calculated against placebo as shown for ** and *** at each time point. Significance testing was performed as part of a post-hoc analysis. Improvement of chorea symptoms in the valbenazine treated group were noted as early as week 2 and continued to improve over the treatment course. This improvement over placebo at all study visits was significant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, in some embodiments, “pharmaceutically acceptable salt” refers to acid addition salts with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002 which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, for example, sodium, potassium, lithium, ammonium, calcium, magnesium, iron, zinc, manganese, aluminum hydroxides, carbonates, bicarbonates, phosphates, and the like; particularly preferred are the ammonium, potassium, sodium, calcium, and magnesium hydroxides, carbonates, bicarbonates, or phosphates. Organic bases from which may be used to prepare salts include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “about” means±20% of the stated value, and includes more specifically values of ±10%, ±5%, ±2% and ±1% of the stated value.

As used herein, “co-administer” and “co-administration” and variants thereof mean the administration of at least two drugs (e.g., valbenazine and a co-therapeutic agent) to a patient either sequentially, simultaneously, approximately simultaneously, or consequently proximate in time to one another (e.g., within the same day, or week or period of 30 days, or sufficiently proximate that each of the at least two drugs can be simultaneously detected in the blood plasma). In some embodiments, the two drugs are administered sequentially in any order. In some embodiments, the two drugs are administered simultaneously or approximately simultaneously. In some embodiments, the two drugs are administered sequentially.

When co-administered, two or more active agents can be co-formulated as part of the same composition or administered as separate formulations. This also may be referred to herein as “concomitant” administration or variants thereof.

As used herein, “adjusting administration,” “altering administration,” “adjusting dosing,” or “altering dosing” are all equivalent and mean tapering off, reducing, or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, “baseline” refers to the period of time just prior to initiation of therapy. The patient's condition just prior to initiation of therapy can be referred to as the patient's baseline condition.

As used herein, “Clinical Global Impression of Change” or CGI-C refers to a rating instrument based on a 7-point scale (range: 1=very much improved to 7-very much worse) that can be used to rate the overall global improvement of chorea since the initiation of study drug dosing. This scale is a modification of a scale developed by the Psychopharmacology Research Branch of the National Institute of Mental Health to rate the subject's overall improvement in clinical disorder and provides a global evaluation of improvement over time from the clinician's perspective.

As used herein, “deutetrabenazine” may be referred to as (RR, SS)-1, 3, 4, 6, 7, 11b-hexahydro-9, 10-di(methoxyd3)-3-(2-methylpropyl)-2H-benzo[a]quinolizin-2-one.

Deutetrabenazine is a racemic mixture containing the following compounds:

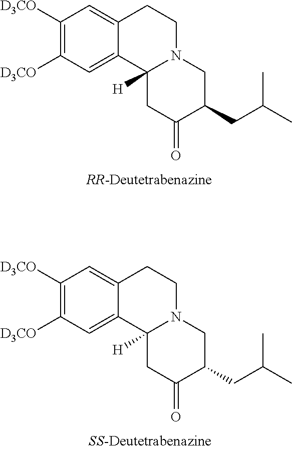

Deutetrabenazine (or do-tetrabenazine) as disclosed in U.S. Pat. No. 8,524,733 is administered resulting in an appropriate concentration over a specified period of time of metabolite (+)a-3-isobutyl-9,10-d6-dimethoxy-1, 3, 4,6,7,11bhexahydro-2H-pyrido[2, 1-a]isoquinolin-2-ol (deuterated (+)a-HTBZ) or deuterated (+)β-HTBZ in the plasma). The d₆-tetrabenazine may be administered by a variety of methods including the formulations as disclosed in PCT Publication WO 2014/047167, the disclosure of which is incorporated herein by reference in its entirety.

As used herein, dihydrotetrabenazine may be referred to as 2-hydroxy-3-(2-methylpropyl)-1,3,4,6,7,11b-hexahydro-9,10-dimethoxy-benzo(a)quinolizine. The compound has three chiral centers and hence can, theoretically, exist in a total of eight isomeric forms as shown below:

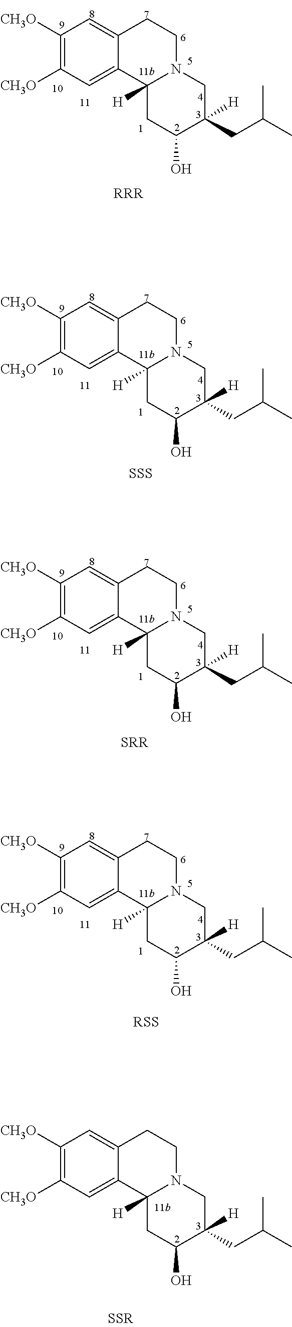

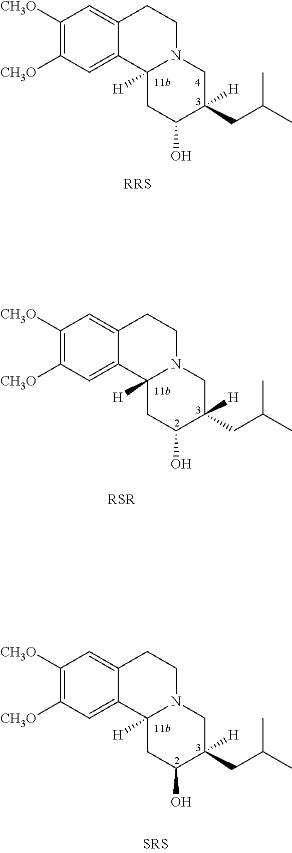

The synthesis and characterization of the eight isomers is described by Sun et al. (2011) Eur. J. Med. Chem. 1841-1848.

As used herein, a “dose” means the measured quantity of an active agent to be taken at one time by a patient. In certain embodiments, wherein the active agent is not valbenazine free base, the quantity is the molar equivalent to the corresponding amount of valbenazine free base. For example, often a drug is packaged in a pharmaceutically acceptable salt form, for example valbenazine ditosylate, and the dosage for strength refers to the mass of the molar equivalent of the corresponding free base, valbenazine. As an example, 73 mg of valbenazine tosylate is the molar equivalent of 40 mg of valbenazine free base.

As used herein, “dosing regimen” means the dose of an active agent taken at a first time by a patient and the interval (time or symptomatic) at which any subsequent doses of the active agent are taken by the patient such as from about 20 to about 160 mg once daily, e.g., about 20, about 40, about 60, about 80, about 100, about 120, or about 160 mg once daily. The additional doses of the active agent can be different from the dose taken at the first time.

As used herein, a “dosage” is the prescribed administration of a specific amount, number, and frequency of doses over a specific period of time.

As used herein, “effective amount” and “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “isotopic variant” means a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴° C.), nitrogen-13 (¹³N), nitrogen-14 (¹⁴N), nitrogen-15 (¹⁵N), oxygen-14 (¹⁴O), oxygen-15 (¹⁵⁰), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (¹⁹Br), bromine-81 (⁸¹Br), iodine-123 (¹²³I), iodine-125 (¹²⁵¹), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), and oxygen-18 (¹⁸O). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), and oxygen-15 (¹⁵O). It will be understood that, in a compound as provided herein, any hydrogen can be ²H, as example, or any carbon can be ¹³C, as example, or any nitrogen can be ¹⁵N, as example, and any oxygen can be ¹⁸O, as example, where feasible according to the judgment of one of skill in the art. In certain embodiments, an “isotopic variant” of a compound contains an unnatural proportion of deuterium.

With regard to the compounds provided herein, when a particular atomic position is designated as having deuterium or “D” or “d,” it is understood that the abundance of deuterium at that position is substantially greater than the natural abundance of deuterium, which is about 0.015%. A position designated as having deuterium typically has a minimum isotopic enrichment factor of, in certain embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium position. The isotopic enrichment of the compounds provided herein can be determined using conventional analytical methods known to one of ordinary skill in the art, including mass spectrometry, nuclear magnetic resonance spectroscopy, and crystallography.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications set forth in 21 CFR 208, and other applicable regulations, which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “Neuro-QoL” is a set of self-report measures that assesses the health-related quality of life (HRQOL) of adults and children with neurological disorders. Neuro-QoL Upper Extremity Function scale measures one's ability across fine motor and activities of daily living involving digital, manual and reach-related function and self-care. Neuro-QoL Lower Extremity Function scale measures one's ability to carry out various activities involving the trunk region and increasing degrees of bodily movement, ambulation, balance or endurance. See, National Institute of Neurological Disorders and Stroke (NINDS). User Manual for the Quality of Life in Neurological Disorders (Neuro-QoL) Measures, Version 2.0, March 2015, which is incorporated herein by reference in its entirety.

As used herein, “patient” or “individual” or “subject” means a mammal, including a human, for whom or which therapy is desired, and generally refers to the recipient of the therapy.

As used herein, “Patient Global Impression of Change (PGI-C)” refers to a rating instrument wherein subjects evaluate the change in their chorea symptoms since initiation of study drug dosing by choosing one of 7 responses (very much improved, much improved, minimally improved, not changed, minimally worse, much worse, and very much worse).

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug Administration. “Pharmacologically active” (or “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, tosylic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

The phrase “an isotopic variant thereof; or a pharmaceutically acceptable salt thereof” as used herein has the same meaning as the phrase “an isotopic variant; or a pharmaceutically acceptable salt of the compound referenced therein; or an isotopic variant; or a pharmaceutically acceptable salt of an enantiomer or a mixture of enantiomers of the compound referenced therein.”

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

As used herein, “Unified Huntington Disease Rating Scale” or “UHDRS” is the standard clinical assessment tool in Huntington disease. The UHDRS is divided into four domains: motor performance, cognitive function, behavioral abnormalities, and functional abilities. The motor section consists of 31 items assessing oculomotor, bradykinesia/rigidity, dystonia, chorea, and gait/balance. The items are rated from zero to four, with zero indicating normal findings and four indicating severe abnormalities. The range of the Total Motor Score (TMS) is 0 to 124, with higher scores indicating more severe motor impairment. The cognitive component includes the verbal fluency test, the symbol digit modalities test, and the Stroop test (color naming, word reading, and interference). Lower scores indicate worse cognitive performance. The behavioral assessment measures the frequency and severity of 11 items, which are rated from zero (almost never/absent) to four (almost always/severe). The items assess depression, anxiety, aggression, psychosis, and other behavioral abnormalities. The behavioral score ranges from 0 to 88, with higher scores indicating more severe psychiatric abnormalities. The functional domain comprises three components, namely the total functional capacity (TFC), the functional assessment scale (FAS), and the independence scale (IS). The TFC consists of five items (occupation, finances, domestic chores, activities of daily living, and care level) and ranges from 0 to 13. The FAS includes 25 yes/no questions about common daily tasks (range 0-25). The Independence Score (IS) measures the level of independence by one single score between 10 and 100. For all functional scores, lower scores indicate a worse function. See, Huntington Study Group, Unified Huntington Disease Rating Scale: reliability and consistency. Mov Disord. 1996; 11:136-142, which is incorporated by reference in its entirety.

As used herein, “VMAT2” refers to human vesicular monoamine transporter isoform 2, an integral membrane protein that acts to transport monoamines, particularly neurotransmitters such as dopamine, norepinephrine, serotonin, and histamine, from cellular cytosol into synaptic vesicles.

As used herein, the terms “VMAT2 inhibitor,” “inhibit VMAT2,” or “inhibition of VMAT2” refer to the ability of a compound disclosed herein to alter the function of VMAT2. A VMAT2 inhibitor may block or reduce the activity of VMAT2 by forming a reversible or irreversible covalent bond between the inhibitor and VMAT2 or through formation of a noncovalently bound complex. Such inhibition may be manifest only in particular cell types or may be contingent on a particular biological event. The terms and/or phrases “VMAT2 inhibitor,” “inhibit VMAT2,” or “inhibition of VMAT2” also refer to altering the function of VMAT2 by decreasing the probability that a complex forms between a VMAT2 and a natural substrate.

As used herein, “tetrabenazine” may be referred to as 1,3,4,6,7,11b-hexahydro-9,10-dimethoxy-3-(2-methylpropyl)-2H-benzo[a]quinolizin-2-one. The compound has chiral centers at the 3- and 11b-carbon atoms and hence can, theoretically, exist in a total of four isomeric forms as shown below:

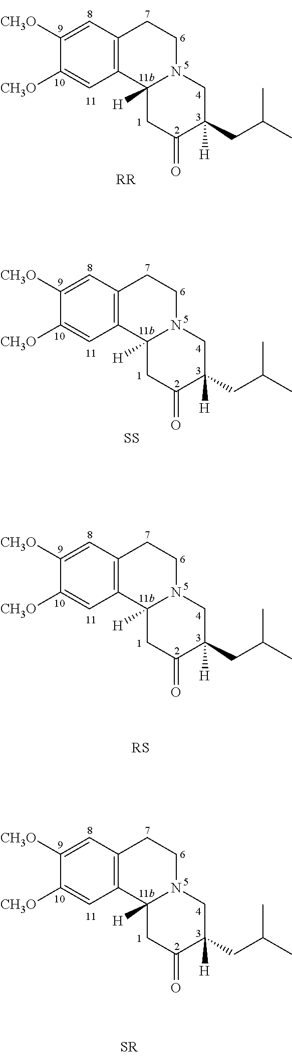

Commercially available tetrabenazine is a racemic mixture of the RR and SS isomers. Tetrabenazine may be administered by a variety of methods including the formulations disclosed in PCT Publications WO 2010/018408, WO 2011/019956, and WO 2014/047167, the disclosure of each of which is incorporated herein by reference in its entirety.

As used herein, “up-titration” of a compound refers to increasing the amount of a compound to achieve a therapeutic effect that occurs before dose-limiting intolerability for the patient. Up-titration can be achieved in one or more dose increments, which may be the same or different.

As used herein, “valbenazine” may be referred to as(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester; or as L-valine, (2R,3R,11bR)-1,3,4,6,7,11b-hexahydro-9,10-dimethoxy-3-(2-methylpropyl)-2H-benzo[a]quinolizin-2-yl ester or as NBI-98854 and has the following chemical structure:

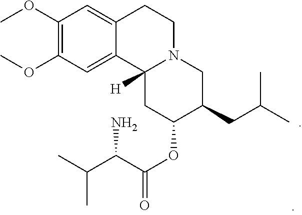 [SMILES: COc1cc2c(cc1OC)[C@H]1C[C@@H](OC(=O)[C@@H](N)C(C)C)[C@H](CC(C)C)CN1CC2]

A formulation of valbenazine: 4-toluenesulfonate (1:2) (referred to herein as “valbenazine ditosylate”) has been previously reported in the FDA approved drug label under the trade name INGREZZA®.

Valbenazine can be prepared according to U.S. Pat. Nos. 8,039,627 and 8,357,697, the disclosure of each of which is incorporated herein by reference in its entirety. In certain embodiments, the valbenazine for use in the compositions and methods provided herein is in polymorphic Form I as disclosed in U.S. Pat. No. 10,0659,52, the disclosure of which is incorporated herein by reference in its entirety.

### Methods of Use

Provided is a method for the treatment of chorea associated with Huntington disease, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof.

In certain embodiments, prior to administration, the subject has a diagnosis of motor manifest HD, i.e., a UHDRS-motor score >5.

In certain embodiments, prior to administration, the subject has a genetic diagnosis of HD with an expanded CAG repeat (≥37) in huntingtin (HTT) gene.

In certain embodiments, prior to administration, the subject has a total maximal chorea score ≥8.

In certain embodiments, prior to administration, the subject has a Total Functional Capacity (TFC) score ≥5.

In certain embodiments, the method is effective to result in an improvement in any one or more of the following:
Unified Huntington Disease Rating Scale total maximal chorea (TMC);
Clinical Global Impression of Change (CGI-C) response status;
Patient Global Impression of Change (PGI-C) response status;
Quality of Life in Neurological Disorders Upper Extremity Function; and
Quality of Life in Neurological Disorders Neuro-QOL Lower Extremity Function.

In certain embodiments, the method is effective to result in an improvement in Unified Huntington Disease Rating Scale total maximal chorea (TMC). In certain embodiments, the improvement in TMC is a change from baseline of at least about 2, at least about 3, at least about 4, or at least about 5. In certain embodiments, the improvement in TMC is a change from baseline of at least 2, at least 3, at least 4, or at least 5. In certain embodiments, the improvement in TMC is a change from baseline to week 2 of at least about 2, such as at least 2. In certain embodiments, the improvement in TMC is a change from baseline to week 4 of at least about 4, such as at least 4. In certain embodiments, the improvement in TMC of at least about 3 after 12 weeks of treatment.

In certain embodiments, the method is effective to result in an improvement in Clinical Global Impression of Change (CGI-C) response status, wherein subjects whose score is either a 1 (“very much improved”) or a 2 (“much improved”) in CGI-C were classified as responders. In certain embodiments, at least about 9% of subjects were classified as responders. In certain embodiments, at least about 10%, such as at least about 15%, at least about 20%, at least about 25%, or at least about 30% of subjects were classified as responders. In certain embodiments, at least about 25%, or at least about 30% of subjects were classified as responders after 12 weeks of treatment.

In certain embodiments, the method is effective to result in an improvement in Patient Global Impression of Change (PGI-C) response status, wherein subjects whose score is either a 1 (“very much improved”) or a 2 (“much improved”) in PGI-C were classified as responders. In certain embodiments, at least about 20%, such as at least about 25%, at least about 30%, at least about 35%, at least about 40%, at least about 50%, at least about 55%, were classified as responders. In certain embodiments, at least about 25% of subjects were classified as responders after 12 weeks of treatment.

In certain embodiments, the method is effective to result in an improvement in Quality of Life in Neurological Disorders Upper Extremity Function and/or an improvement in Quality of Life in Neurological Disorders Neuro-QoL Lower Extremity Function.

Valbenazine can be administered according to the methods disclosed in U.S. Pat. Nos. 10,857,137; 10,874,648; 10,912,771; 10,940,141; 10,952,997; 10,857,148; and 10,993,941, the disclosure of each of which is incorporated herein by reference in its entirety.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester. In a further embodiment, the initial dosage is 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the patient is administered a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester with an initial dosage of 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base, and the dosage is increased by 20 mg increments every two weeks to a recommended dosage. In a further embodiment, the recommended dosage is 80 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base. In another embodiment, the recommended dosage is 60 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the patient is monitored for one or more adverse reactions selected from somnolence, akathisia, fatigue, urticaria, rash, nausea and middle insomnia. In a further embodiment, the one or more adverse reactions is selected from somnolence, urticaria and rash.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered via a titration scheme that comprises the up-titration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester over a period of no more than about six weeks until an optimized dose is administered.

In certain embodiments, the titration scheme comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof, or a pharmaceutically acceptable salt thereof at an initial dose equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for about two weeks, provided that the subject tolerates the initial dose and that the subject has not had an adequate response, increasing the dose and administering the increased dose to the subject.

In certain embodiments, the increased dose is equivalent to about 60 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, the titration scheme further comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof at the increased dose for about two weeks.

In certain embodiments, if the subject does not tolerate the increased dose, the optimized dose is the initial dose.

In certain embodiments, if the subject tolerates the increased dose and if the subject has had an adequate response, the optimized dose is the increased dose.

In certain embodiments, the methods further comprise administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof to the subject.

In certain embodiments, if the subject tolerates the increased dose and if the subject has not had an adequate response, the method further comprises increasing the dose.

In certain embodiments, the further increased dose is equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

In certain embodiments, if the subject does not tolerate the further increased dose, the optimized dose is the increased dose.

In certain embodiments, if the subject tolerates the further increased dose and if the subject has had an adequate response, the optimized dose is the further increased dose.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered in the afternoon or evening.

In some embodiments, administration of valbenazine orally once daily (QD) as adjunctive treatment in subjects with chorea associated with Huntington disease, wherein the valbenazine is administered at an initial dose for a period of time followed by a scheduled dose increase. In certain embodiments, the period of time for the initial dose is a week. In certain embodiments, the initial dose is equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base. In certain embodiments, the increased dose is equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base. In certain embodiments, the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in a solid dosage form.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is orally administered.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in the form of a capsule.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily or twice daily.

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily.

In certain embodiments, the therapeutically effective amount is an amount equivalent to from about 10 mg to about 90 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to from about 20 mg to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 20 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 40 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 60 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In certain embodiments, the therapeutically effective amount is an amount equivalent to about 80 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

In some embodiments, the VMAT2 inhibitor is chosen from valbenazine, or a pharmaceutically acceptable salt and/or isotopic variant thereof. In some embodiments, the VMAT2 inhibitor is valbenazine, or a pharmaceutically acceptable salt thereof. In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a free base.

In some embodiments, the VMAT2 inhibitor is a valbenazine tosylate salt. In some embodiments, the VMAT2 inhibitor is a ditosylate salt of valbenazine. In certain embodiments, the tosylate salt is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt of structural Formula (I):

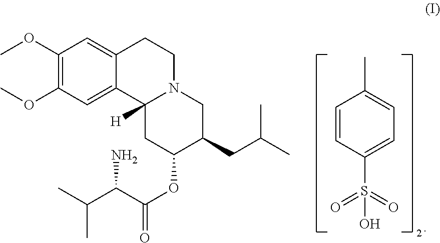

In certain embodiments, the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is in crystalline form.

In some embodiments, crystalline Form I has a DSC thermogram comprising an endothermic event with an onset temperature of about 240° C. and a peak at about 243° C.

In certain embodiments, the crystalline form of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is Form I of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt having a differential scanning calorimetric (DSC) peak temperature within 2% of 243° C.

In certain embodiments, the DSC peak temperature is within 1% of 243° C.

In certain embodiments, the DSC peak temperature is within 0.5% of 243° C.

In various embodiments, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) has an X-ray diffraction pattern. In some embodiments, the X-ray diffraction pattern of Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3, 17.9, and 19.7°. In some embodiments, the X-ray powder diffraction pattern of Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3, 17.9, or 19.7°. In another embodiment, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3° and 19.7°. In another embodiment, crystalline Form I of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) includes an XRP diffraction peak at two-theta angles of approximately 6.3°.

In some embodiments, crystalline Form I has one or more characteristic XRP diffraction peaks at two-theta angles of approximately 6.3° and approximately 19.7°. In certain embodiments, crystalline Form I has one or more characteristic XRP diffraction peaks at two-theta angles of approximately 6.3°, approximately 17.9°, and approximately 19.7°. In some embodiments, crystalline Form I has one or more characteristic XRP diffraction peaks at two-theta angles of approximately 6.3°, approximately 17.9°, approximately 19.7°, and approximately 22.7°. In certain embodiments, crystalline Form I has one or more characteristic XRP diffraction peaks at two-theta angles of approximately 6.3°, approximately 15.6°, approximately 17.9°, approximately 19.7°, and approximately 22.7°. In some embodiments, crystalline Form I has one or more characteristic XRP diffraction peaks at two-theta angles of approximately 6.3°, approximately 15.6°, approximately 16.6°, approximately 17.9°, approximately 19.7°, and approximately 22.7°. In certain embodiments, the crystalline form has an X-ray powder diffraction (XRPD) pattern comprising a peak at a two-theta angle of 6.3°+0.2°. In certain embodiments, the crystalline form has an X-ray powder diffraction (XRPD) pattern comprising a peak at a two-theta angle of 17.9°+0.2°. In certain embodiments, the crystalline form has an X-ray powder diffraction (XRPD) pattern comprising a peak at a two-theta angle of 19.7°+0.2°.

In yet another embodiment, crystalline Form I has a thermal gravimetric analysis (TGA) plot comprising a mass loss of less than about 0.4% when heated from about 25° C. to about 140° C.

In various embodiments, crystalline Form I has a gravimetric vapor system (GVS) plot. In some embodiments, crystalline Form I exhibit a mass increase of about 1% when subjected to an increase in relative humidity from about 0% to about 95% relative humidity. In certain embodiments mass gained upon adsorption is lost when the relative humidity (RH) is decreased back to about 0% RH. In still another embodiment, crystalline Form I is stable upon exposure to about 25° C. and about 60% relative humidity. In yet another embodiment, crystalline Form I is stable upon exposure to about 25° C. and about 60% relative humidity for about 24 months. Also in another embodiment, crystalline Form I is stable upon exposure to about 25° C. and about 60% relative humidity for about 3 months. In still another embodiment, crystalline Form I is stable upon exposure to about 25° C. and about 92% relative humidity. In another embodiment, crystalline Form I is stable upon exposure to about 40° C. and about 75% relative humidity. In another embodiment, crystalline Form I is stable upon exposure to about 40° C. and about 75% relative humidity for about 6 months. In another embodiment, crystalline Form I is stable upon exposure to about 40° C. and about 75% relative humidity for about 3 months.

In certain embodiments, crystalline form of Formula I in Form I may contain no less than about 95%, no less than about 97%, no less than about 98%, no less than about 99%, or no less than about 99.5% by weight of the salt of Formula I. The crystalline form may also contain no less than about 90%, no less than about 95%, no less than about 98%, no less than about 99%, or no less than 99.5% by weight of crystal Form I.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt.

In certain embodiments, the crystalline form has a purity of no less than 98% by weight of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt.

In certain embodiments, the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt; and has an X-ray powder diffraction (XRPD) pattern comprising peaks at two-theta angles of 6.3°+0.2°, 17.9°=0.2°, and 19.7°+0.2°.

In certain embodiments, crystalline Form I has an aqueous solubility of about 17.58, about 18.58, about 19.58, about 26.75, about 26.87, about 26.96, about 27.06, about 27.75, about 27.87, about 27.97, about 28.06, about 28.75, about 28.87, about 28.97, about 29.06, about 27.45, about 28.45, about 29.45, about 30.61, about 31.61, about 32.61, about 32.17, about 32.98, about 33.17, about 33.98, about 34.17, about 34.35, about 34.98, about 35.35, about 36.35 mg/mL. In certain embodiments, crystalline Form I has an aqueous solubility of about 31.61 and about 33.17 at approximately pH 1.2; about 28.45 and about 27.97 at approximately pH 3; about 28.06 and about 27.77 at approximately pH 4; about 18.58 and about 27.87 at approximately pH 5; about 33.98 and about 35.35 at approximately pH 6.8.

In certain embodiments, crystalline Form I may contain no greater than about 0.1%, no greater than about 0.11%, no greater than about 0.12%, no greater than about 0.13%, no greater than about 0.14%, no greater than about 0.15%, no greater than about 0.16%, no greater than about 0.17%, no greater than about 0.18%, no greater than about 0.19%, no greater than about 0.2%, no greater than about 0.21%, no greater than about 0.22%, no greater than about 0.23%, no greater than about 0.24%, no greater than about 0.25%, no greater than about 0.26%, no greater than about 0.27%, no greater than about 0.28%, no greater than about 0.29%, no greater than about 0.3%, no greater than about 0.31%, no greater than about 0.32%, no greater than about 0.33%, no greater than about 0.34%, no greater than about 0.35%, no greater than about 0.36%, no greater than about 0.37%, no greater than about 0.38%, no greater than about 0.39%, no greater than about 0.4%, no greater than about 0.5%, no greater than about 0.6%, no greater than about 0.7%, no greater than about 0.8%, no greater than about 0.9%, no greater than about 1%, no greater than about 2%, no greater than about 3%, no greater than about 4%, or no greater than about 5% water by weight.

In certain embodiments Form I may be characterized by particle analysis. In certain embodiments, a sample of Form I comprises particles having rhomboid crystal morphology. In yet another embodiment, a sample of Form I comprises particles of about 100, about 90, about 80, about 70, about 60, about 50, about 40, about 30, about 20, about 10, about 5 μM in length. In some embodiments, a sample of Form I comprises particles of about 70, about 60, about 40, about 20, about 10 μM in length. In other embodiments, a sample of Form I comprises particles of about 69.39, about 56.22, about 34.72, about 17.84, about 10.29 μM in length.

In certain embodiments, the crystalline form has a D90 particle size of about 70 μM in length.

In certain embodiments, the crystalline form has a D10 particle size of about 10 μM in length.

In some embodiments, the VMAT2 inhibitor is an isotopic variant that is L-valine, (2R,3R,11bR)-1,3,4,6,7,11b-hexahydro-9,10-di(methoxy-d3)-3-(2-methylpropyl)-2H-benzo[a]quinolizin-2-yl ester or a pharmaceutically acceptable salt thereof.

In some embodiments, the VMAT2 inhibitor is tetrabenazine (9,10-dimethoxy-3-isobutyl-1,3,4,6,7,11b-hexahydro-2H-pyrido[2,1-a]isoquinolin-2-one), or a pharmaceutically acceptable salt and/or isotopic variant thereof. In some embodiments, tetrabenazine is chosen from the RR, SS, RS, and SR isomers of tetrabenazine, and mixtures thereof. In some embodiments, tetrabenazine is a mixture of the RR and SS isomers.

In some embodiments, the VMAT2 inhibitor is deutetrabenazine.

In some embodiments, the VMAT2 inhibitor is chosen from dihydrotetrabenazine (2-hydroxy-3-(2-methylpropyl)-1,3,4,6,7,11b-hexahydro-9,10-dimethoxy-benzo (a) quinolizine), or a pharmaceutically acceptable salt and/or isotopic variant thereof. In some embodiments, dihydrotetrabenazine is chosen from the RRR, SSS, SSRR, RSS, SSR, RRS, RSR, and SRS isomers of dihydrotetrabenazine, and mixtures thereof. In some embodiments, the VMAT2 inhibitor is the RRR isomer ((+)-a-3-isobutyl-9,10-dimethoxy-1,3,4,6,7,11b-hexahydro-2H-pyrido[2,1-a]isoquinolin-2-ol), or a pharmaceutically acceptable salt and/or isotopic variant thereof.

In some embodiments, provided herein is method for treatment of chorea associated with Huntington disease, wherein the patient is also being administered a strong cytochrome P450 2D6 (CYP2D6) inhibitor, comprising:
orally administering once daily to the patient a vesicular monoamine transport 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for treatment of chorea associated with Huntington disease, wherein the patient is a cytochrome P450 2D6 (CYP2D6) poor metabolizer, comprising:
orally administering once daily to the patient a vesicular monoamine transporter 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount of equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for treatment of chorea associated with Huntington disease, comprising:
(a) orally administering to the patient a therapeutically effective amount of a vesicular monoamine transport 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof;
(b) subsequently determining that the patient is a cytochrome P450 2D6 (CYP2D6) poor metabolizer; and
(c) reducing dosage of the VMAT2 inhibitor administered to the patient to an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for treatment of chorea associated with Huntington disease, comprising:
determining if the patient is a poor metabolizer of cytochrome P450 2D6 (CYP2D6); and
if the patient is a poor metabolizer of cytochrome P450 2D6 (CYP2D6), then orally administering to the patient a first therapeutically effective amount of a vesicular monoamine transport 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, wherein the first therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester; or
if the patient is not a poor metabolizer of cytochrome P450 2D6 (CYP2D6), then orally administering to the patient a second therapeutically effective amount of a vesicular monoamine transport 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, wherein the second therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester for one week, and subsequently administering an increased amount of the VMAT2 inhibitor after one week.

In some embodiments, provided herein is a method for treatment of chorea associated with Huntington disease, comprising:
(a) orally administering to the patient a therapeutically effective amount of a vesicular monoamine transport 2 (VMAT2) inhibitor which is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, wherein the therapeutically effective amount is an amount equivalent to about 40 mg once daily as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
(b) subsequently determining that the patient is a poor metabolizer of cytochrome P450 2D6 (CYP2D6); and
(c) administering the same therapeutically effective amount of the VMAT2 inhibitor to the patient.

In some embodiments, provided herein is a method for treatment of chorea associated with Huntington disease, wherein the patient is a cytochrome P450 2D6 (CYP2D6) poor metabolizer, comprising:
orally administering once daily to the patient a therapeutically effective amount of a vesicular monoamine transporter 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof.

In some embodiments, provided herein is a method of administering a vesicular monoamine transporter (VMAT2) inhibitor chosen from valbenazine and (+)-a-3-isobutyl-9,10-dimethoxy-1,3,4,6,7,11b-hexahydro-2H-pyrido[2,1-a]isoquinolin-2-ol, or a pharmaceutically acceptable salt thereof, or an isotopic variant thereof, to a patient in need thereof, wherein the patient is being treated with a strong cytochrome P450 3A4 (CYP3A4) inducer, comprising:
discontinuing treatment of the strong CYP3A4 inducer and then administering the VMAT2 inhibitor to the patient, thereby avoiding the use of the VMAT2 inhibitor in combination with the strong CYP3A4 inducer.

In some embodiments, provided herein is a method for treatment of chorea associated with Huntington disease in a patient in need thereof, wherein the patient is being administered a strong cytochrome P450 3A4 (CYP3A4) inducer, comprising:
discontinuing treatment of the strong CYP3A4 inducer, and then
orally administering once daily to the patient a therapeutically effective amount of a vesicular monoamine transporter 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and a pharmaceutically acceptable salt thereof, thereby avoiding the concomitant use of the VMAT2 inhibitor with the strong CYP3A4 inducer.

In some embodiments, provided herein is a method for treatment of chorea associated with Huntington disease, wherein the patient is also being administered a strong cytochrome P450 3A4 (CYP3A4) inhibitor, comprising:
orally administering once daily to the patient a vesicular monoamine transporter 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof, in an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

In some embodiments, provided herein is a method for treatment of chorea associated with Huntington disease in a patient, comprising:
(a) orally administering to the patient a therapeutically effective amount of a vesicular monoamine transporter 2 (VMAT2) inhibitor chosen from(S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester and pharmaceutically acceptable salts thereof;
(b) subsequently determining that the patient is being administered a strong cytochrome P450 3A4 (CYP3A4) inhibitor; and
(c) reducing dosage of the VMAT2 inhibitor administered to the patient to an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily.

In some embodiments, provided herein is a method for treatment of chorea associated with Huntington disease in a patient, comprising:
(a) orally administering to the patient a therapeutically effective amount of a vesicular monoamine transporter 2 (VMAT2) inhibitor which is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, wherein the therapeutically effective amount is an amount equivalent to about 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester once daily;
(b) subsequently determining that the patient is being administered a strong cytochrome P450 3A4 (CYP3A4) inhibitor; and
(c) administering the same therapeutically effective amount of the VMAT2 inhibitor to the patient.

In some embodiments, provided herein is a method for treatment of chorea associated with Huntington disease, wherein the patient is also being co-administered digoxin, comprising:
(a) administering to the patient a therapeutically effective amount of a vesicular monoamine transport 2 (VMAT2) inhibitor chosen from(S)-2-amino-3-methyl-butyric acid (2R,3R,11bR)-3 sobutyl-9,10-dimethoxy-1,3,4,6,7,11b-hexahydro-2H-pyrido[2,1-a]isoquinolin-2-yl ester, (+)-a-3-isobutyl-9,10-dimethoxy-1,3,4,6,7,11b-hexahydro-2H-pyrido[2,1-a]isoquinolin-2-ol, and pharmaceutically acceptable salts and isotopic variants thereof,
(b) monitoring the digoxin concentration in the patient's blood; and
(c) reducing the dose of digoxin when the digoxin exposure in the patient's blood is increased as compared with the digoxin level in a patient who is administered digoxin alone.

In some embodiments, provided herein is a method for treatment of chorea associated with Huntington disease, wherein the patient is also in need of treatment with digoxin, the method comprising:
orally administering to the patient a therapeutically effective amount of a vesicular monoamine transport 2 (VMAT2) inhibitor which is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester ditosylate, and
administering the digoxin to the patient at a reduced dose to compensate for the expected increase in exposure resulting from co-administration of the digoxin and the VMAT2 inhibitor,
wherein the reduced dose is relative to what the patient would be administered if the patient is not being administered the VMAT2 inhibitor.

In some embodiments, a method for treatment of chorea associated with Huntington disease, comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof, wherein the subject is also being administered at least one co-therapeutic agent for the treatment of chorea associated with Huntington disease is provided herein.

In some embodiments, the VMAT2 inhibitor is administered via a titration scheme that comprises the up-titration of the VMAT2 inhibitor over a period of no more than about six weeks until an optimized dose is administered.

In some embodiments, the titration scheme comprises administering the VMAT2 inhibitor at an initial dose equivalent to about 40 mg of valbenazine free base once daily for about two weeks, provided that the patient tolerates the initial dose and that the patient has not had an adequate response, increasing the dose, and administering the increased dose to the patient.

In some embodiments, the increased dose is equivalent to about 60 mg of valbenazine free base once daily.

In some embodiments, the titration scheme further comprises administering the VMAT2 inhibitor at said increased dose for about two weeks.

In some embodiments, if the patient does not tolerate the increased dose, the optimized dose is the initial dose.

In some embodiments, if the patient tolerates the increased dose and if the patient has had an adequate response, the optimized dose is the increased dose.

In some embodiments, the method further comprises administering the optimized dose of the VMAT2 inhibitor to the patient.

In some embodiments, if the patient tolerates the increased dose and if the patient has not had an adequate response, the method further comprises increasing the dose.

In some embodiments, the further increased dose is equivalent to about 80 mg of valbenazine free base once daily.

In some embodiments, if the patient does not tolerate the further increased dose, the optimized dose is the increased dose.

In some embodiments, if the patient tolerates the further increased dose and if the patient has had an adequate response, the optimized dose is the further increased dose.

In some embodiments, the method further comprises administering the optimized dose of the VMAT2 inhibitor to the patient.

In one embodiment, provided herein is a method for the treatment inhibition of human vesicular monoamine transporter isoform 2 (VMAT2), comprising administering to a subject a therapeutically effective amount of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate) (Formula I) in an amorphous form, or crystalline Form I, II, III, IV, V, or VI; or an isotopic variant thereof; or solvate thereof.

In one embodiment, provided herein is a method for the treatment inhibition of human vesicular monoamine transporter isoform 2 (VMAT2), comprising administering to a subject a therapeutically effective amount of (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate dihydrochloride (Formula II) in an amorphous form, or crystalline Form I, or II; or an isotopic variant thereof; or solvate thereof.

### PHARMACEUTICAL COMPOSITIONS

Also provided is a composition for treating a patient in need of a vesicular monoamine transport 2 (VMAT2) inhibitor chosen from valbenazine and (+)-a-3-isobutyl-9,10-dimethoxy-1,3,4,6,7,11b-hexahydro-2H-pyrido[2,1-a]isoquinolin-2-ol, or a pharmaceutically acceptable salt and/or isotopic variant thereof, comprising a therapeutically effective amount of the VMAT2 inhibitor.

In certain embodiments, the composition is for treating a neurological or psychiatric disease or disorder. In certain embodiments, the neurological or psychiatric disease or disorder is chorea associated with Huntington disease.

In certain embodiments, the composition is administered in an amount equivalent to from about 20 mg to about 120 mg of valbenazine free base of the VMAT2 inhibitor. In certain embodiments, the composition is administered in an amount equivalent to about 20 mg of valbenazine free base of the VMAT2 inhibitor. In certain embodiments, the composition is administered in an amount equivalent to about 40 mg of valbenazine free base of the VMAT2 inhibitor. In certain embodiments, the composition is administered in an amount equivalent to about 80 mg of valbenazine free base of the VMAT2 inhibitor. In certain embodiments, the composition is administered in an amount equivalent to about 60 mg of valbenazine free base of the VMAT2 inhibitor. In certain embodiments, the composition is administered in an amount equivalent to about 120 mg of valbenazine free base of the VMAT2 inhibitor.

In certain embodiments, the composition is administered for a first period of time in a first amount of the VMAT2 inhibitor and then the amount is increased to a second amount. In certain embodiments, the first period of time is a week. In certain embodiments, the first amount is equivalent to about 40 mg of valbenazine free base. In certain embodiments, the second amount is equivalent to about 80 mg of valbenazine free base.

Also provided herein is a pharmaceutical composition for use in treating neurological or psychiatric disease or disorders, comprising the VMAT2 inhibitor as an active pharmaceutical ingredient, in combination with one or more pharmaceutically acceptable carriers or excipients.

The choice of excipient, to a large extent, depends on factors, such as the particular mode of administration, the effect of the excipient on the solubility and stability of the active ingredient, and the nature of the dosage form.

The pharmaceutical compositions provided herein may be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein may be administered alone, or in combination with one or more other compounds provided herein, one or more other active ingredients. The pharmaceutical compositions provided herein may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Oral Administration

The pharmaceutical compositions provided herein may be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant. Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation. The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems.

The pharmaceutical compositions provided herein may be provided as noneffervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide. Coloring and flavoring agents can be used in all of the above dosage forms. The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H2-receptor antagonists.

### Dosages

In the treatment of conditions, disorders or diseases associated with VMAT2 inhibition, an appropriate dosage level generally is about 0.001 to 100 mg per kg patient body weight per day (mg/kg per day), about 0.01 to about 80 mg/kg per day, about 0.1 to about 50 mg/kg per day, about 0.5 to about 25 mg/kg per day, or about 1 to about 20 mg/kg per day, which may be administered in single or multiple doses. Within this range the dosage may be 0.005 to 0.05, 0.05 to 0.5, or 0.5 to 5.0, 1 to 15, 1 to 20, or 1 to 50 mg/kg per day. In certain embodiments, the dosage level is about 0.001 to 100 mg/kg per day.

In certain embodiments, the dosage level is about from 20 to 100 mg/kg per day. In certain embodiments, the dosage level is about 0.01 to about 40 mg/kg per day. In certain embodiments, the dosage level is about 0.1 to about 80 mg/kg per day. In certain embodiments, the dosage level is about 0.1 to about 50 mg/kg per day. In certain embodiments, the dosage level is about 0.1 to about 40 mg/kg per day. In certain embodiments, the dosage level is about 0.5 to about 80 mg/kg per day. In certain embodiments, the dosage level is about 0.5 to about 40 mg/kg per day. In certain embodiments, the dosage level is about 0.5 to about 25 mg/kg per day. In certain embodiments, the dosage level is about 1 to about 80 mg/kg per day. In certain embodiments, the dosage level is about 1 to about 75 mg/kg per day. In certain embodiments, the dosage level is about 1 to about 50 mg/kg per day. In certain embodiments, the dosage level is about 1 to about 40 mg/kg per day. In certain embodiments, the dosage level is about 1 to about 25 mg/kg per day. In certain embodiments, the dosage level is about 1 to about 20 mg/kg per day.

In certain embodiments, the dosage level is about from 5.0 to 150 mg per day, and in certain embodiments from 10 to 100 mg per day. In certain embodiments, the dosage level is about 80 mg per day. In certain embodiments, the dosage level is about 40 mg per day.

For oral administration, the pharmaceutical compositions can be provided in the form of tablet or capsule containing 1.0 to 1,000 mg of the active ingredient, particularly about 1, about 5, about 10, about 15, about 20, about 25, about 30, about 40, about 45, about 50, about 75, about 80, about 100, about 150, about 200, about 250, about 300, about 400, about 500, about 600, about 750, about 800, about 900, and about 1,000 mg of the active ingredient for the symptomatic adjustment of the dosage to the patient to be treated. In certain embodiments, the pharmaceutical compositions can be provided in the form of tablet or capsule containing about 100 mg of the active ingredient. In certain embodiments, the pharmaceutical compositions can be provided in the form of tablet or capsule containing about 80 mg of the active ingredient. In certain embodiments, the pharmaceutical compositions can be provided in the form of tablet or capsule containing about 60 mg of the active ingredient. In certain embodiments, the pharmaceutical compositions can be provided in the form of tablet or capsule containing about 50 mg of the active ingredient. In certain embodiments, the pharmaceutical compositions can be provided in the form of tablet or capsule containing about 40 mg of the active ingredient. In certain embodiments, the pharmaceutical compositions can be provided in the form of tablet or capsule containing about 20 mg of the active ingredient. In certain embodiments, the pharmaceutical compositions can be provided in the form of tablet or capsule containing about 25 mg of the active ingredient. In certain embodiments, the pharmaceutical compositions can be provided in the form of tablet or capsule containing about 20 mg of the active ingredient. The compositions may be administered on a regimen of 1 to 4 times per day, including once, twice, three times, and four times per day. In certain embodiments, the pharmaceutical compositions are administered on a regimen of once per day.

It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds provided herein may also be combined or used in combination with other agents useful in the treatment, prevention, or amelioration of one or more symptoms of the diseases or conditions for which the compounds provided herein are useful.

Such other agents, or drugs, may be administered, by a route and in an amount commonly used thereof, simultaneously, or sequentially with the compounds provided herein. When compounds provided herein are used contemporaneously with one or more other drugs, a pharmaceutical composition containing such other drugs in addition to the compounds provided herein may be utilized but is not required. Accordingly, the pharmaceutical compositions provided herein include those that also contain one or more other active ingredients or therapeutic agents, in addition to the compounds provided herein.

The weight ratio of the compounds provided herein to the second active ingredient may be varied and will depend upon the effective dose of each ingredient. Generally, an effective dose of each will be used. Thus, for example, when the compounds provided herein are used in combination with the second drug, or a pharmaceutical composition containing such other drug, the weight ratio of the particulates to the second drug may range from about 1,000:1 to about 1:1,000, or about 200:1 to about 1:200.

Combinations of the particulates provided herein and other active ingredients will generally also be within the aforementioned range, but in each case, an effective dose of each active ingredient should be used.

In certain embodiments, the VMAT2 inhibitor is administered orally.

In certain embodiments, the VMAT2 inhibitor is administered in the form of a capsule.

In certain embodiments, the VMAT2 inhibitor is administered with or without food.

Exemplary embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1

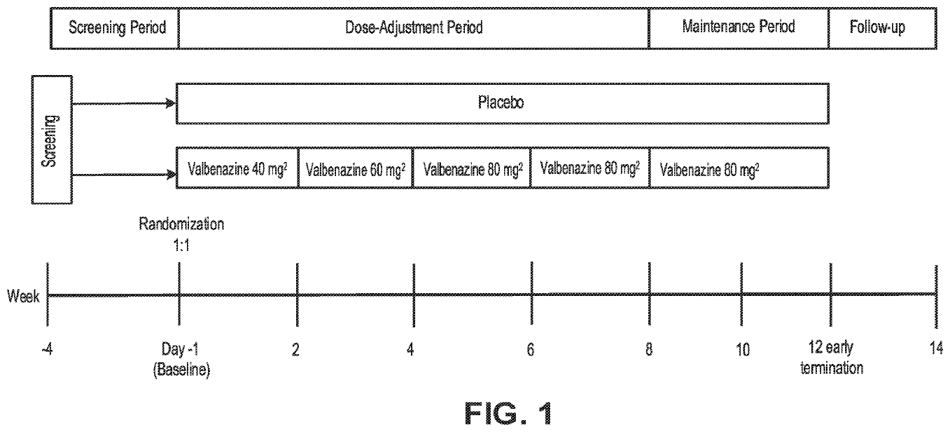

A Phase 3, randomized, double-blind, placebo-controlled study to evaluate the efficacy, safety, and tolerability of once-daily valbenazine in subjects with HD was conducted. The study included a 4-week screening period, an 8-week dose-adjustment period, a 4-week maintenance period, and a final study visit 2 weeks following the final dose of study drug. A shows a schematic of the study design is shown in FIG. 1.

Valbenazine was supplied as orally administered capsules containing 20 or 40 mg of valbenazine (free base equivalents as the ditosylate salt). Subjects must swallow the capsules with approximately 4 ounces of water or other liquid, with or without food.

The maximum doses during each week of the treatment period are shown in Table 1 below.

TABLE 1

| | | | | | Maintenance |
|---|---|---|---|---|---|
| | Dose Adjustment Period | | | | Period |
| Week | 1-2 | 3-4 | 5-6 | 7-8 | 9-12 |
| Arm 1 | Placebo | Placebo | Placebo | Placebo | Placebo |
| Arm 2 | Valbenazine | Valbenazine | Valbenazine | Valbenazine | Valbenazine |
| | 40 mg | 60 mg | 80 mg | 80 mg | 80 mg |

During the dose-adjustment period, the investigator could increase a subject's dose to the next dose level if, in the investigator's opinion, the subject has tolerated the study drug at the current dose. Doses were adjusted in a blinded manner; subjects receiving placebo will undergo the dose-adjustment process but will continue to receive placebo. Dose increases were allowed at visits at the end of Weeks 2, 4, and 6. If the subject had not tolerated the current dose, the investigator could decrease the subject's dose at any time during the dose adjustment period (the 40 mg dose can be decreased to 20 mg). Doses were to be decreased 1 dose level at a time and subjects could have multiple dose decreases during the dose-adjustment period; subjects who were unable to tolerate the 20 mg dose could remain in the study but study drug dosing was discontinued. Subjects who had a dose decrease could re-escalate during the dose adjustment period if the investigator considered that the dose increase would be reasonably tolerated.

During the maintenance period (beginning after the Week 8 visit through the end of Week 12), the subject's dose was maintained. If the subject could not tolerate the maintenance dose, the investigator could reduce the subject's dose a single time by 1 dose level (unless the subject is receiving 20 mg); if the subject could not tolerate the lower dose, he/she could remain in the study but study drug dosing was discontinued.

During the dose-adjustment and maintenance periods, the investigator could assess that a dose level is not tolerated if a subject experienced an adverse event (AE) that is (1) deemed associated with the study drug, and (2) of either moderate or severe intensity, or a serious AE.

The primary endpoint for the study was change from baseline to maintenance in the Unified Huntington Disease Rating Scale total maximal chorea (TMC).

Secondary endpoints included:
Clinical Global Impression of Change (CGI-C) response status (score ≥2 of “very much improved” or “much improved”) at Week 12;
Patient Global Impression of Change (PGI-C) response status (score ≥2 of “very much improved” or “much improved”) at Week 12;
Change from baseline to Week 12 in the Quality of Life in Neurological Disorders Upper Extremity Function; and
Change from baseline to Week 12 in the Neuro-QoL Lower Extremity Function.

Exploratory endpoints included:
Wearable movement sensors (in select sites; optional participation) using MC10 Inc., BIOSTAMP® NPOINT® system
Huntington Disease Health Index
Changes from the screening period baseline to each postbaseline study visit (Weeks 2 through 12) in the TMC based on site assessments
Changes from the screening period baseline to maintenance (the average of the Week 10 and Week 12 assessments) in the TMC based on video recording central rater assessments
CGI-C response at Weeks 2 through 10
PGI-C response at Weeks 2 through 10
The change from baseline to Weeks 4, 8, and 10 in the Neuro-QoL Upper Extremity Function T-score.
The change from baseline to Weeks 4, 8, and 10 in the Neuro-QoL Lower Extremity Function T-score
CGI-S at Weeks 2 through 12
PGI-S at Weeks 2 through 12
SF-36 at Week 12
HD-HI at Week 10 and Week 12
EQ-5D-5L at Week 10 and Week 12
UHDRS scores for motor, behavior, and functional capacity assessment at Weeks 2 through 12
Other UHDRS scores including functional assessment and independence scale at Week 12
AS at Week 12
Change from screening period in physical activity by movement sensors at the Week 10 visit period

Key inclusion criteria included:
Male or female 18 to 75 years old
Clinical diagnosis of Huntington disease with chorea
Genetic diagnosis of Huntington disease with an expanded CAG repeat (≥37) in huntingtin (HTT) gene
Be able to walk, with or without the assistance of a person or device
Total Maximal Chorea score ≥8 at screening and baseline
Total Function Capacity (TFC) score ≥5 at screening
   Participants with a TFC score between 5 and 10 (inclusive) must have a reliable caregiver to ensure study drug administration and attendance at study visits

Exclusion criteria included:
History of prior VMAT2 inhibitor therapy
Difficulty swallowing
Currently pregnant or breastfeeding
History or evidence of long QT syndrome, cardiac tachyarrhythmia, left bundle branch block, atrioventricular block, bradycardia (<60 bpm), or heart failure
Unstable or serious medical or psychiatric illness
Significant risk of suicidal behavior
Current substance dependence or substance (drug) or alcohol abuse
Have an untreated or undertreated psychiatric illness, such as depression
   Participants receiving antidepressant therapy may be enrolled if they have been on a stable dose for at least 8 weeks prior to baseline
Have any of the following laboratory test abnormalities: serum creatinine >1.5×ULN, AST≥2.5×ULN, ALT≥2.5×ULN, GGT≥3.0×ULN, total bilirubin >1.5 mg/dL

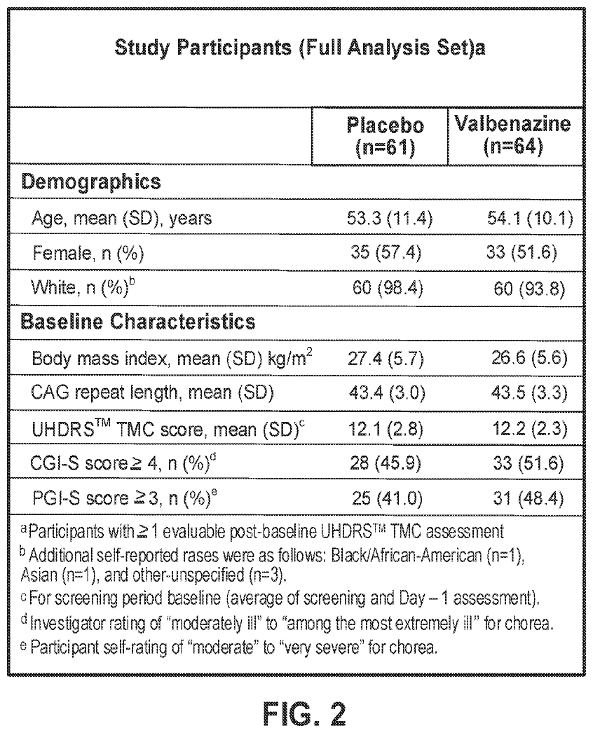

Baseline characteristics for the subjects were as follows (Table 2), with additional subject information provided in FIG. 2:

TABLE 2

| | Placebo | | Valbenazine | | All Subjects | |
|---|---|---|---|---|---|---|
| | (N = 61) | | (N = 64) | | (N = 125) | |
| Height (cm), mean (SD) | 167.67 | (11.57) | 169.79 | (9.24) | 168.76 | (10.45) |
| Weight (kg), mean (SD) | 77.63 | (19.88) | 76.4 | (16.25) | 77 | (18.05) |
| BMI (kg/m²), mean (SD) | 27.4 | (5.70) | 26.55 | (5.58) | 26.96 | (5.63) |
| CAG Repeat Length, mean (SD) | 43.4 | (3.0) | 43.5 | (3.3) | 43.4 | (3.2) |
| CYP2D6 Phenotype Status | | | | | | |
| Poor Metabolizer | 6 | (9.8) | 3 | (4.7) | 9 | (7.2) |
| Intermediate Metabolizer | 22 | (36.1) | 25 | (39.1) | 47 | (37.6) |
| Normal Metabolizer | 30 | (49.2) | 30 | (46.9) | 60 | (48.0) |
| Ultra-rapid Metabolizer | 1 | (1.6) | 5 | (7.8) | 6 | (4.8) |
| Not Collected | 2 | (3.3) | 1 | (1.6) | 3 | (2.4) |
| Baseline Montreal Cognitive | 24.2 | (3.2) | 22.9 | (4.3) | 23.5 | (3.9) |
| Assessment Score (MoCA), | | | | | | |
| mean (SD) | | | | | | |
| Swallowing Disturbance | 5.2 | (6.2) | 4.9 | (6.2) | 5 | (6.2) |
| Questionnaire (SDQ) Total | | | | | | |
| Score, mean (SD) | | | | | | |
| Screening Period Baseline | 12.07 | (2.78) | 12.23 | (2.32) | 12.16 | (2.55) |
| TMC - On-Site | | | | | | |
| Assessments, mean (SD) | | | | | | |
| Baseline CGI-S score | | | | | | |
| category | | | | | | |
| <4 | 33 | (54.1) | 31 | (48.4) | 64 | (51.2) |
| >=4 | 28 | (45.9) | 33 | (51.6) | 61 | (48.8) |
| Baseline PGI-S score | | | | | | |
| category | | | | | | |
| <3 | 36 | (59.0) | 33 | (51.6) | 69 | (55.2) |
| >=3 | 25 | (41.0) | 31 | (48.4) | 56 | (44.8) |

Data shown are n (%) unless otherwise specified

Provided below in Table 3 is a summary of results for the primary and secondary endpoints.

TABLE 3

| | | | | | 95% | |
|---|---|---|---|---|---|---|
| | | | | | Confidence | |
| | Efficacy | Placebo | Valbenazine | Difference | Interval | P-value |
| Primary | TMC | −1.44 | −4.6 | −3.16 | (−4.37, −1.95) | <0.0001 |
| | CFSPB @ | | | | | |
| | Maintenance, | | | | | |
| | LS Mean | | | | | |
| Secondary | CGI-C @ | 13.2% | 42.9% | 29.7% | (11%, 45%) | 0.0007 |
| | week 12, % | | | | | |
| | responders | | | | | |
| | PGI-C @ | 26.4% | 52.7% | 26.3% | (6%, 44%) | 0.0062 |
| | week 12, % | | | | | |
| | responders | | | | | |
| | Neuro-QoL | −3 | −1.58 | 1.42 | (−1.46, 4.31) | 0.3305 |
| | Upper | | | | | |
| | Extremity | | | | | |
| | Function T | | | | | |
| | Score CFB | | | | | |
| | @ week 12, | | | | | |
| | LS Mean | | | | | |
| | Neuro-QoL | 0.6 | −0.27 | −0.88 | (−3.20, 1.43) | 0.4517 |
| | Lower | | | | | |
| | Extremity | | | | | |
| | Function T | | | | | |
| | Score CFB | | | | | |
| | @ week 12, | | | | | |
| | LS Mean | | | | | |

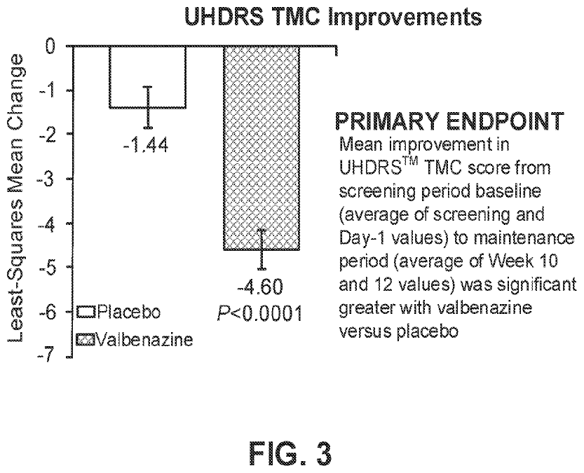

The average TMC score during the baseline period was 12.2 (SD 2.3). Valbenazine demonstrated a statistically significant improvement in chorea severity with a placebo-adjusted mean reduction of 3.2 units in TMC score vs. placebo (LS Mean change from baseline −4.6 vs.-1.4; P<0.0001; see e.g., FIG. 3). Statistically significant secondary endpoints of Clinical Global Impression of Change (CGI-C) Response Status and Patient Global Impression of Change (PGI-C) Response Status favored valbenazine treatment. Neuro-QOL upper and lower extremity physical function endpoints did not meet statistical significance.

Overall, there were comparable rates of TEAEs in both treatment groups, comparable rates and low incidence of SAE and AE leading to discontinuation of study treatment, and no worsening of suicidal ideation or behavior in the valbenazine arm.

The most commonly reported AE was somnolence (valbenazine: 15.6%, placebo: 3.2%). No suicidal behavior or worsening of suicidal ideation was observed in the valbenazine treated subjects.

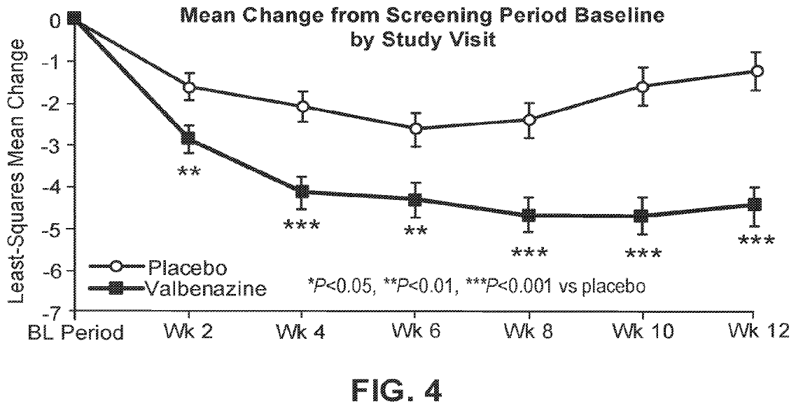

Once-daily administration of valbenazine was associated with a significant improvement in chorea (see e.g., FIGS. 3-4). Valbenazine was well tolerated, and treatment emergent adverse events observed were consistent with the known safety profile of valbenazine.

Provided below in Table 4 is a summary of results for the exploratory endpoints.

TABLE 4

| Exploratory | |
|---|---|
| Efficacy | |
| Endpoint | Summary of Results |
| TMC Score: | The observed values of the investigator-assessed TMC score |
| Change from | decreased from screening period baseline for the valbenazine group |
| screening period | relative to the placebo group at each study visit through Week 12, |
| baseline to each | and LS mean differences between treatment groups (valbenazine |
| study visit based | minus placebo) were as follows: Week 2 (−1.26 [95% CI: −2.18, −0.34], |
| on-site | nominal p = 0.0077), Week 4 (−2.05 [95% CI: −3.12, −0.98], |
| assessments | nominal p = 0.0002), Week 6 (−1.69 [95% CI: −2.83, −0.55], nominal |
| | p = 0.0041), Week 8 (−2.29 [95% CI: −3.46, −1.13], nominal |
| | p = 0.0002); Week 10 (−3.10 [95% CI: −4.36, −1.84], nominal |
| | p < 0.0001), and Week 12 (−3.22 [95% CI: −4.53, −1.91], nominal |
| | p < 0.0001). At Week 14, 2 weeks after discontinuation of study |
| | treatment, absolute TMC scores in the valbenazine group were |
| | similar to those of the placebo group. |
| TMC Score: | The LS mean change from screening period baseline to maintenance |
| Change from | in TMC scores as assessed by the central video raters was −3.51 |
| screening period | (95% CI: −4.32, −2.69) for the valbenazine group and −0.92 (95% |
| baseline to | CI: −1.77, −0.07) for the placebo group, with a treatment difference |
| maintenance | of −2.59 (95% CI: −3.76, −1.41); p < 0.0001 (nominal), and was consistent |
| based on central | with and supportive of the primary efficacy analysis of TMC based |
| video rater | on on-site assessments. |
| assessments | |
| CGI-C response | CGI-C response rate was higher for the valbenazine group relative to |
| status at Weeks 2 | the placebo group at each visit through Week 10. Differences |
| through 10 | between treatment groups (valbenazine minus placebo) were as |
| | follows: Week 2 (7.71% [95% CI: −0.93, 17.87], nominal p = 0.1157), |
| | Week 4 (20.42% [95% CI: 7.53, 33.00], nominal p = 0.0012), Week 6 |
| | (20.14% [95% CI: 4.04, 35.89], nominal p = 0.0151), Week 8 |
| | (34.50% [95% CI: 16.46, 49.76], nominal p = 0.0001), and Week 10 |
| | (24.80% [95% CI: 6.55, 41.18], nominal p = 0.0065). |
| PGI-C response | PGI-C response rate was higher for the valbenazine group relative to |
| status at Weeks 2 | the placebo group at each visit. Differences between treatment |
| through 10 | groups (valbenazine minus placebo) were observed at Weeks 2 |
| | through 10 as follows: Week 2 (18.52% [95% CI: 5.48, 31.41], |
| | nominal p = 0.0042), Week 4 (18.57% [95% CI: 3.79, 32.70], nominal |
| | p = 0.0120), Week 6 (23.08% [95% CI: 5.60, 39.04], nominal |
| | p = 0.0064), Week 8 (26.44% [95% CI: 7.70, 42.49], nominal |
| | p = 0.0037), and Week 10 (16.94% [95% CI: −1.37, 34.63], nominal |
| | p = 0.0747). |
| Neuro-QoL Upper | The Neuro-QoL Upper Extremity Function T-Score was stable for |
| Extremity | the valbenazine group and decreased from baseline for the placebo |
| Function T-Score | group at Weeks 4, 8, and 10, consistent with the outcome of the |
| change from | secondary efficacy endpoint of Neuro-QoL Upper Extremity |
| baseline to Weeks | Function T-Score at Week 12. |
| 4, 8, and 10 | |
| Neuro-QoL | No notable differences between treatment groups were seen in the |
| Lower Extremity | Neuro-QoL Lower Extremity Function T-Score at Weeks 4, 8, and |
| Function T-Score | 10, consistent with the outcome of the secondary efficacy endpoint |
| change from | of Neuro-QoL Lower Extremity Function T-Score at Week 12. |
| baseline to Weeks | |
| 4, 8, and 10 | |
| CGI-S at Week 2 | Most subjects were considered mildly or moderately ill at baseline, |
| through Week 12 | and the proportions were similar between treatment groups. At Week |
| | 12, an improvement in CGI-S toward not at all ill, borderline ill, or |
| | mildly ill was observed for the valbenazine group compared with the |
| | placebo group. |
| PGI-S at Week 2 | At baseline, most subjects rated their chorea as mild or moderate. At |
| through Week 12 | Week 12, the proportion of valbenazine-treated subjects who had no |
| | chorea symptoms increased to about 27% from about 9% at baseline, |
| | the proportion with mild symptoms remained consistent (about |
| | 45%), and the proportion with moderate symptoms decreased from |
| | about 41% to about 29%. No valbenazine-treated subject had severe |
| | or very severe symptoms at Week 12. |
| | The proportions of placebo-treated subjects did not vary considerably |
| | across categories from baseline to Week 12. |
| SF-36 at Week 12 | No notable differences were observed at Week 12 between treatment |
| | groups in the SF-36 subscales and summary scales (physical health |
| | component or mental health component). In an exploratory analysis |
| | using an ANCOVA model for the change from baseline to Week 12 |
| | in SF-36 subscales and summary scales, no statistically significant |
| | differences were noted between treatment groups. |
| | No worsening from baseline was observed for the valbenazine group |
| | in any subscale or summary scales. |
| HD-HI at Week | Numerical decreases from baseline to Weeks 10 and 12 in HD-HI |
| 10 and Week 12 | subscales were noted for the valbenazine group compared with |
| | placebo for mobility (Week 10: −6.319 valbenazine vs −4.131 |
| | placebo; Week 12: −4.208 vs −2.708), involuntary movements (Week |
| | 10: −12.220 vs −9.584; Week 12: −11.195 vs −4.270), and hand and |
| | arm function (Week 10: −7.154 vs −1.628; Week 12: −7.169 |
| | vs −1.392); however, baseline values were slightly higher for the |
| | valbenazine group for mobility and hand and arm function. |
| | An exploratory analysis using an ANCOVA model was performed |
| | for the change from baseline in HD-HI subscales. In the involuntary |
| | movements subscale, the LS mean change from baseline to Week 12 |
| | was −11.068 (95% CI: −15.424, −6.712) for the valbenazine group |
| | and −4.406 (95% CI: −8.934, 0.121) for the placebo group, with an |
| | LS mean difference between treatment groups of −6.662 (95% |
| | CI: −12.945, −0.379); p = 0.0379 (nominal). |
| | Changes from baseline to Weeks 10 and 12 in the fatigue and |
| | swallowing function subscales were unremarkable for the |
| | valbenazine group. No meaningful changes in the HD-HI total score |
| | were noted from baseline at Weeks 10 and 12. |
| EQ-5D-5L at | No notable differences between treatment groups were observed |
| Week 10 and | from baseline to Weeks 10 and 12 in change in EQ-5D-5L health |
| Week 12 | index score and distribution of scores for each dimension. No shift |
| | toward worsening was observed for the valbenazine group in any |
| | dimension. |
| UHDRS TMS at | The mean TMS decreased from baseline for the valbenazine group |
| Weeks 2 through | compared with the placebo group at each visit through Week 12 |
| 12 | (−7.5 vs −3.2); at Week 14, the change from baseline in TMS was |
| | similar between treatment groups (−3.8 vs −3.1). |
| | An exploratory analysis using an MMRM model demonstrated a |
| | difference in the change from screening period baseline to |
| | maintenance in TMS score between the valbenazine and placebo |
| | groups: LS mean difference −3.99 (95% CI: −6.27, −1.70); p = 0.0008 |
| | (nominal). |
| UHDRS scores | No notable changes were noted from baseline to Week 12 in |
| for cognitive and | UHDRS cognitive and behavior assessments in either treatment |
| behavior | group, indicating stability of these outcomes. |
| assessments and | |
| at Weeks 2 | |
| through 12 | |
| Other UHDRS | No clinically meaningful changes were noted from baseline to Week |
| scores including | 12 in UHDRS functional assessment, independence, and total |
| functional | functional capacity scores in either treatment group. |
| assessment, | |
| independence | |
| scale, and total | |
| functional | |
| capacity at Week | |
| 12 | |
| Anosognosia | No clinically meaningful changes were noted from baseline to Week |
| Scale at Week 12 | 12 in Anosognosia Scale score as assessed by the investigator, |
| | subject, and the difference (subject minus investigator score) for |
| | either treatment group. |

ANCOVA = analysis of covariance; CGI-C = Clinical Global Impression of Change; CGI-S = Clinical Global Impression of Severity; EQ-5D-5L = EuroQol 5 Dimensions 5 Levels; HD-HI = Huntington Disease Health Index; MMRM = mixed-effect model repeated measures; Neuro-QoL = Quality of Life in Neurological Disorders; PGI-C = Patient Global Impression of Change; PGI-S = Patient Global Impression of Severity; SF-36 = Short Form 36 Health Survey; TMC = Total Maximal Chorea; TMS = total motor score; UHDRS = Unified Huntington Disease Rating Scale.

Additional exploratory endpoints are described below.

### Clinical Global Impression of Severity

The investigator rated the severity of a subject's chorea using the CGI-S at scheduled visits on a 7-point scale. The number and percentage of subjects in each CGI-S category was summarized by and treatment group and visit. Shifts in CGI-S scores from baseline were presented in a shift table by treatment and visit.

### Patient Global Impression of Severity

Subjects rated the severity of their chorea at scheduled visits on a 5-point scale. The PGI-S was analyzed using the same methods as the CGI-S.

### Short Form 36 Health Survey

The SF-36 is 36-item questionnaire that measures health on 8 dimensions. Observed values and change from baseline to Week 12 in dimension scores and component summaries were summarized descriptively by treatment group.

### Huntington Disease Health Index

The HD-HI includes 13 subscales that measure 13 individual areas of health in patients with HD. Observed values and change from baseline were summarized descriptively for each subscale and the total instrument score by treatment group and visit.

### EuroQol 5 Dimensions 5 Levels

The EQ-5D-5L contains 5 dimensions (Mobility, Self-Care, Usual Activities, Pain/Discomfort, and Anxiety/Depression), and each dimension has 5 levels (no problems, slight problems, moderate problems, severe problems, and extreme problems). The scores for the 5 dimensions are combined into a 5-digit number that describes the patient's health state. The number and percentage of subjects in each of the 5 levels for each of 5 dimensions at baseline and Weeks 10 and 12 was summarized by treatment group. The EQ VAS score at each time point and change from baseline to Week 10 and Week 12 were summarized by treatment group.

Health states in the EQ-5D-5L were converted into a single index value, where index values are presented in the country-specific value sets. Observed values and change from baseline in EQ-5D-5L index values were summarized by treatment group and visit.

### UHDRS Scores for Motor, Cognitive, Behavior, and Functional Capacity Assessment

The motor portion of the UHDRS (TMS) consists of 15 items that measure the severity of the motor symptoms. The TMS was summarized by treatment group and visit (Weeks 2 through 12) similar to the analysis of the TMC. The cognitive portion of the UHDRS consists of 3 executive function tests to evaluate cognitive performance. The score of individual tests was summarized by treatment group and visit. The behavior portion of the UHDRS consists of 11 items (under 4 subscales: mood, behavior, psychosis, and obsessive) that measure the severity and frequency of behavior symptoms. The frequency and severity of each item is ranked on a 0 to 4 scale. The sum of behavior frequency scores (11 total items, range 0-44), and the sum of behavior frequency-times-severity item scores total item products, range 0-176) was summarized. The behavior milestone questions (items 36 to 40) were summarized by treatment group.

The functional capacity portion of the UHDRS consists of 5 items. The individual items and TFC score were summarized by treatment group and visit (Weeks 2 through 12) similar to the analysis of the TMC.

### UHDRS Scores for Functional Assessment and Independence Scale

The functional assessment portion of the UHDRS consists of 25 questions with binary outcomes (Yes=1, No=0). The total scores defined as the sum of the 25 questions at baseline and Weeks 12 were summarized by treatment group. The independence scale of UHDRS was similarly summarized.

### Example 2: Formulations/Study Medications

The composition of 20, 40, 60, and 80 mg dose strength valbenazine is provided in the Tables 5-6 below.

### Formulation 1: Quantitative Composition of Valbenazine Capsules, 20, 40, 60, and 80 mg (Free Base Equivalent)

TABLE 5

| Component | % (w/w) |
|---|---|
| Valbenazine ditosylate | 40.0 |
| Silicified microcrystalline cellulose | 25.0 |
| Isomalt | 20.0 |
| Hypromellose | 5.0 |
| Partially pregelatinized maize starch | 7.5 |
| Magnesium stearate | 2.5 |
| Total | 100 |
| Hard gelatin capsule | 1 unit |

### Formulation 2: Quantitative Composition of Valbenazine Capsules, 40 mg (Free Base Equivalent)

TABLE 6

| Component | % (w/w) |
|---|---|
| Valbenazine ditosylate | 28.2 |
| Mannitol | 61.8 |
| Partially pregelatinized maize starch | 7.7 |
| Fumed silica | 1.2 |
| Magnesium stearate | 1.0 |
| Total | 100 |
| Hard gelatin capsule | 1 unit |

Although the disclosure has been described with reference to the above examples, it will be understood that modifications and variations are encompassed within the spirit and scope of the disclosure. The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method for the treatment of chorea associated with Huntington disease (HD), comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof;
wherein, prior to the administration, the subject has one or more characteristics selected from the group consisting of:
a diagnosis of motor manifest HD;
a genetic diagnosis of HD with an expanded CAG repeat (≥37) in huntingtin (HTT) gene;
a total maximal chorea score ≥8; and
a Total Functional Capacity (TFC) score ≥5.

2. The method of claim 1, wherein:
prior to administration, the subject has a diagnosis of motor manifest HD.

3. The method of claim 1 or 2, wherein:
prior to administration, the subject has a genetic diagnosis of HD with an expanded CAG repeat (≥37) in huntingtin (HTT) gene.

4. The method of any one of the preceding claims, wherein:
prior to administration, the subject has a total maximal chorea score ≥8.

5. The method of any one of the preceding claims, wherein:
prior to administration, the subject has a Total Functional Capacity (TFC) score ≥5.

6. The method of any one of the preceding claims, wherein the method is effective to result in an improvement in any one or more of the following:
Unified Huntington Disease Rating Scale total maximal chorea (TMC);
Clinical Global Impression of Change (CGI-C) response status;
Patient Global Impression of Change (PGI-C) response status;
Quality of Life in Neurological Disorders Upper Extremity Function; and
Quality of Life in Neurological Disorders Neuro-QoL Lower Extremity Function.

7. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered via a titration scheme that comprises the up-titration of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester over a period of no more than about six weeks until an optimized dose is administered.

8. The method of claim 7, wherein the titration scheme comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof at an initial dose equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily for about two weeks, provided that the patient tolerates the initial dose and that the patient has not had an adequate response, increasing the dose and administering the increased dose to the subject.

9. The method of claim 8, wherein the increased dose is equivalent to about 60 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

10. The method of claim 8, wherein the increased dose is equivalent to about 80 mg of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

11. The method of any one of claims 7-10, wherein the titration scheme further comprises administering the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof at the increased dose for about two weeks.

12. The method of any one of claims 7-10, wherein if the subject does not tolerate the increased dose, the optimized dose is the initial dose.

13. The method of claim 11, wherein if the subject tolerates the increased dose and if the subject has had an adequate response, the optimized dose is the increased dose.

14. The method of claim 12 or 13, further comprising administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof to the subject.

15. The method of claim 11, wherein if the subject tolerates the increased dose and if the subject has not had an adequate response, the method further comprises increasing the dose.

16. The method of claim 15, wherein the further increased dose is equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

17. The method of claim 15 or 16, wherein if the subject does not tolerate the further increased dose, the optimized dose is the increased dose.

18. The method of claim 15 or 16, wherein if the subject tolerates the further increased dose and if the subject has had an adequate response, the optimized dose is the further increased dose.

19. The method of claim 17 or 18, further comprising administering the optimized dose of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof to the subject.

20. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in a solid dosage form.

21. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is orally administered.

22. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is in the form of a capsule.

23. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered daily.

24. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily or twice daily.

25. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered once daily.

26. The method of claim 25, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is administered in the afternoon or evening.

27. The method of any one of the preceding claims, wherein the therapeutically effective amount is an amount equivalent to from about 10 mg to about 90 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

28. The method of any one of the preceding claims, wherein the therapeutically effective amount is an amount equivalent to from about 20 mg to about 80 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

29. The method of any one of the preceding claims, wherein the therapeutically effective amount is an amount equivalent to about 20 mg of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester free base.

30. The method of any one of claims 1-28, wherein the therapeutically effective amount is an amount equivalent to about 40 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

31. The method of any one of claims 1-28, wherein the therapeutically effective amount is an amount equivalent to about 60 mg of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

32. The method of any one of claims 1-28, wherein the therapeutically effective amount is an amount equivalent to about 80 mg of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester free base.

33. The method of any one of claims 1-28, wherein the therapeutically effective amount is an amount equivalent to about 40 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

34. The method of any one of claims 1-28, wherein the therapeutically effective amount is an amount equivalent to about 60 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

35. The method of any one of claims 1-28, wherein the therapeutically effective amount is an amount equivalent to about 80 mg/day of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base.

36. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a free base.

37. The method of any one of claims 1-35, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester is a salt.

38. The method of claim 37, wherein the salt is a tosylate salt.

39. The method of claim 38, wherein the tosylate salt is (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester tosylate salt of structural Formula (I):

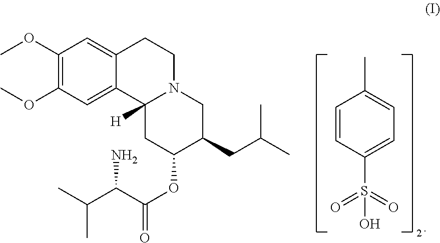

40. The method of any one of the preceding claims, wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is in crystalline form.

41. The method of claim 40, wherein the crystalline form of the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt, is Form I of (S)-2-amino-3-methyl-butyric acid (2R,3R,11bR)-3-isobutyl-9,10-dimethoxy-1,3,4,6,7,11b-hexahydro-2H-pyrido[2, I-a]isoquinolin-2-yl ester tosylate salt having a differential scanning calorimetric (DSC) peak temperature within 2% of 243° C.

42. The crystalline form of claim 40 or 41, wherein the DSC peak temperature is within 1% of 243° C.

43. The crystalline form of any one of claims 40-42, wherein the DSC peak temperature is within 0.5% of 243° C.

44. The crystalline form of any one of claims 40-43, wherein the crystalline form has an X-ray powder diffraction (XRPD) pattern comprising a peak at a two-theta angle of 6.3°+0.2°.

45. The crystalline form of any one of claims 40-44, wherein the crystalline form has an X-ray powder diffraction (XRPD) pattern comprising a peak at a two-theta angle of 17.9°+0.2°.

46. The crystalline form of any one of claims 40-45, wherein the crystalline form has an X-ray powder diffraction (XRPD) pattern comprising a peak at a two-theta angle of 19.7°+0.2°.

47. The crystalline form of any one of claims 40-46, wherein the crystalline form is stable upon exposure to about 25° C. and about 60% relative humidity.

48. The crystalline form of any one of claims 40-47, wherein the crystalline form has a D90 particle size of about 70 μM in length.

49. The crystalline form of any one of claims 40-48, wherein the crystalline form has a D10 particle size of about 10 UM in length.

50. The crystalline form of any one of claims 40-49, wherein the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, I-a]isoquinolin-2-yl ester tosylate salt.

51. The crystalline form of any one of claims 40-50, wherein the crystalline form has a purity of no less than 98% by weight of (S)-2-amino-3-methyl-butyric acid (2R,3R,11bR)-3-isobutyl-9,10-dimethoxy-1,3,4,6,7,11b-hexahydro-2H-pyrido[2, I-a]isoquinolin-2-yl ester tosylate salt.

52. The crystalline form of any one of claims 40-51, wherein the crystalline form has a purity of no less than 97% by weight of (S)-2-amino-3-methyl-butyric acid (2R,3R,11bR)-3-isobutyl-9,10-dimethoxy-1,3,4,6,7,11b-hexahydro-2H-pyrido[2, I-a]isoquinolin-2-yl ester tosylate salt; and has an X-ray powder diffraction (XRPD) pattern comprising peaks at two-theta angles of 6.3°+0.2°, 17.9°+0.2°, and 19.7°+0.2°.

53. A method for the treatment of chorea associated with Huntington disease (HD), comprising administering to a subject in need thereof a therapeutically effective amount of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof;
wherein the (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant thereof; or a pharmaceutically acceptable salt thereof is a ditosylate salt of (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, and
wherein the ditosylate salt of (S)-2-amino-3-methyl-butyric acid (2R,3R,11bR)-3-isobutyl-9,10-dimethoxy-1,3,4,6,7,11b-hexahydro-2H-pyrido[2, 1-a]isoquinolin-2-yl ester is administered with an initial dosage of 40 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily, and the dosage is increased by 20 mg increments every two weeks to a recommended dosage.

54. The method of claim 53, wherein the recommended dosage is 60 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

55. The method of claim 53, wherein the recommended dosage is 80 mg as measured by (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester free base once daily.

56. The method of any one of claims 53-55, wherein prior to administration, the subject has a diagnosis of motor manifest HD.

57. The method of any one of claims 53-56, wherein prior to administration, the subject has a genetic diagnosis of HD with an expanded CAG repeat (≥37) in huntingtin (HTT) gene.

58. The method of any one of claims 53-57, wherein prior to administration, the subject has a total maximal chorea score ≥8.

59. The method of any one of claims 53-58, wherein prior to administration, the subject has a Total Functional Capacity (TFC) score ≥5.

60. The method of any one of claims 53-59, wherein the method is effective to result in an improvement in any one or more of the following:
Unified Huntington Disease Rating Scale total maximal chorea (TMC);
Clinical Global Impression of Change (CGI-C) response status;
Patient Global Impression of Change (PGI-C) response status;
Quality of Life in Neurological Disorders Upper Extremity Function; and
Quality of Life in Neurological Disorders Neuro-QoL Lower Extremity Function.
